(19) United States
(12) Patent Application Publication
(10) Pub. No.: US 2021/0251249 A1
(43) Pub. Date: Aug. 19, 2021

(54) Systems and Methods of Sanitizing Powdered Food Product

(71) Applicant: LacNation LLC, Sheridan, WY (US)

(72) Inventors: Jeremy Peter Mueller, Provo, UT (US); Quinn Weston Everett, Provo, UT (US)

(21) Appl. No.: 17/245,612

(22) Filed: Apr. 30, 2021

Related U.S. Application Data
(63) Continuation-in-part of application No. 17/000,129, filed on Aug. 21, 2020.
(60) Provisional application No. 62/894,254, filed on Aug. 30, 2019.

(51) Int. Cl. A23C 3/07; A23L 3/28; A23L 3/00; A61L 2/10

(52) U.S. Cl. CPC A23C 3/076; A23L 3/28; A23C 1/04; A61L 2/10; A23V 2002/00; A23L 3/001

(57) ABSTRACT

This application is directed to systems and methods of creating sterilized powdered food product. Specific examples regarding breast milk are described whereby the milk is powderized and placed into a vat before it is passed along to a sterilization chamber in which it is fluidized and sterilized by UV-C light. Once powdered food product has been sufficiently sterilized, it can be loaded into syringes for distribution. Embodiments of the inventive subject matter are designed to maintain nutritional value of the powdered food at levels that were not previously possible, opening new opportunities for storing and distribution of, e.g., donor breast milk.

This application is a continuation-in-part of and claims priority to U.S. patent application Ser. No. 17/000,129 filed Aug. 21, 2020, which claims priority to U.S. Provisional Patent Application No. 62/894,254 filed Aug. 30, 2019. All extrinsic materials identified in this application are incorporated by reference in their entirety.

### FIELD OF THE INVENTION

The field of the invention is the sterilization of a powdered food substance.

### BACKGROUND

The background description includes information that may be useful in understanding the present invention. It is not an admission that any of the information provided in this application is prior art or relevant to the presently claimed invention, or that any publication specifically or implicitly referenced is prior art.

For a wide variety of reasons, there is always demand for powderized foods. Breast milk, for example, is needed particularly in neonatal intensive care units. Although the background description focuses on the breast milk use case, these deficiencies exist across a spectrum of powdered food markets. Unlike cow's milk prepared for the normal consumer market, donated breast milk intended for neonates must be prepared in a way that not only renders it safe for consumption, but also maximizes nutrient retention. Nutrient retention in the context of neonatal care not only pertains to macronutrient and micronutrient availability but the bioactivity of key enzymes present in milk, e.g. lysozyme, lactoferrin, sIgA etc., as well. Heat-based pasteurization techniques damages the structure, and therefore, function of these nutrients to varying degrees.

Whereas dairy products are typically created for well-baby and adult markets, donor breast milk is a food that becomes a therapeutic when used for pre-term infants in neonatal intensive care units (NICUs). Such infants rely on the biologically active nutrients contained within the milk to grow and thrive. Many of these infants do not have access to their own mother's milk, or, in certain cases, their own mother's milk is nutritionally insufficient to power their growth, giving rise to a need for pasteurized, screened donor milk products.

The problem within the donor breast milk industry concerns that of balancing the three key customer demands of creating a product: safety, nutrient retention, and affordability. Preterm infants are particularly vulnerable to infection and therefore bacterial load in donor breast milk must be significantly reduced and viruses must ideally be completely inactivated. Key immunoprotective proteins do not work if they have been denatured in the process of pasteurization, rendering traditional pasteurization an unfit technique for these narrow situations. Depending on the type of pasteurization used, milk may need to be kept frozen until bedside use, dramatically increasing shipping costs compared to its shelf-stable counterparts.

In 2020, hospitals routinely incur costs of between $7 and $13 per ounce for donor breast milk—higher than many less ideal alternatives. While studies show that using donor breast milk lowers the overall cost of care for preterm infants, some NICUs may struggle with convincing hospital administrators to look beyond the up-front cost at the potential savings. The result is that these hospitals either do not use donor breast milk or donor breast milk use is limited to a select demographic of NICU patients (e.g., 32 weeks' gestation and under).

Existing products successfully satisfy two of the three key consumer demands. With safety an absolute must, this means products either compromise on nutrient retention to favor a lower cost or vice versa. Even so, all products compromise on nutrient retention to some degree. This is due to the use of various heat-based processes (e.g., Holder's, high-temperature short-time, retort) and hospitals seem more willing to sacrifice nutrition than cost or safety. Heat denatures proteins and speeds up biochemical degradation reactions, and, in doing so, causes the loss of the biologically active proteins to varying degrees. For example, in retort processing, milk is made “commercially sterile” and shelf-stable but at the expense of the bioactivity of key immunoprotective nutrients (e.g., sIgA). The protein may still exist in the milk but no longer plays a role in protecting the infant from infection. Donor breast milk banks use thermal processing to pasteurize milk for lack of a better alternative. Milk banks that use pasteurization techniques that cause relatively less damage to the nutrients require milk to remain frozen post-processing up through delivery. Breast milk banks are then restricted to local delivery due to the high cost of shipping frozen milk.

To maximize nutrient retention, some employ pasteurization methods that do not completely reduce certain strains of bacteria (e.g., B. Cereus). To eliminate remaining bacteria, milk is routinely screened post-pasteurization, and any milk that continues to be contaminated with such bacteria is thrown out, leading to increased donor breast milk costs due to waste. Moreover, milk sterilized in this way must be kept frozen until use at the bedside, increasing its cost via cold-storage shipping and the drain on clinician time for thawing it prior to use. Cold-storage shipping becomes an increasing drain on a donor breast milk business's supply chain based on shipping distances.

One way to solve the problems outlined above would be to sterilize unpasteurized, powdered breast milk, but no suitable solutions currently exist. Once these problems are solved, it can become possible to create a low-cost, high-nutritional value powdered breast milk with a long shelf-life to make storage and shipment easier.

Some have made efforts to innovate in this space, but none have developed adequate solutions. For example, U.S. Pat. No. 8,007,847 to Biderman et al. teaches the use of UV light for sterilization of water in reservoirs in the context of handling milk/formula, but Biderman et al. does not contemplate using UV light to sterilize powdered milk. In another example, U.S. Pat. No. 7,572,632 to Fike et al. teaches that irradiation may be accomplished by exposing a powdered media, media supplement, media subgroup or buffer, prior to packaging, to a source of gamma rays or a source of ultraviolet light. But this reference is virtually absent on details about how this process can work, and it makes no mention of breast milk nor how this can be accomplished while retaining nutrients. Neither reference contemplates solutions to the “shadow effect” problem whereby only thin layers of an opaque (or mostly opaque) substance can be exposed to UV light at any given time. Whereas gamma radiation can effectively sterilize dense, opaque substances, UV light is classically limited with powders due to the tendency for the top layer to cast a shadow on deeper layers.

These and all other extrinsic materials discussed in this application are incorporated by reference in their entirety. Where a definition or use of a term in an incorporated reference is inconsistent or contrary to the definition of that term provided in this application, the definition of that term provided in this application applies and the definition of that term in the reference does not apply.

Thus, there remains a need for improved systems and methods directed to sterilization of a powderized food product.

### SUMMARY OF THE INVENTION

The present invention provides apparatuses, systems, and methods directed to sterilization of powdered food product. In one aspect of the inventive subject matter, a sterilization chamber for powdered food product is contemplated, the sterilization chamber comprising: a cylindrical basin configured to receive the powdered food product; a first end wall and a second end wall disposed on each end of the cylindrical basin, wherein the first end wall comprises a door; a transparent tube having a UV-C light tube disposed therein, where the transparent tube is positioned along a central axis of the cylindrical basin; where the transparent tube couples with the first end wall and with the second end wall; a paddle ribbon configured to rotate about the central axis of the cylindrical basin; and a set of UV-C light strips disposed above the cylindrical basin.

In some embodiments, the UV-C light tube and the UV-C light strips are configured to emit light having a wavelength between 100 nm and 400 nm. The UV-C light tube and the UV-C light strips can be configured to apply 20-200 mJ/cm³ of UV-C light to the powdered food product to create sterilized powdered food. In some embodiments, the sterilization chamber also includes a push plate having a shape complementary to the cylindrical basin and a push rod configured to move the push plate, where the push rod and push plate are together configured move the powdered food product or sterilized food product out of the cylindrical basin through the door on the first end wall.

In another aspect of the inventive subject matter, a sterilization chamber for powdered food product is contemplated, the sterilization chamber comprising: a cylindrical basin configured to receive the powdered food product; a first end wall and a second end wall disposed on each end of the cylindrical basin, wherein the first end wall comprises a door; a lid coupled with the first end wall and the second end wall; a transparent tube having a UV-C light tube disposed therein, where the transparent tube is positioned along a central axis of the cylindrical basin; where the transparent tube couples with the first end wall and with the second end wall; a set of UV-C light strips disposed above the cylindrical basin; and a motor coupled with a paddle ribbon, where the paddle ribbon is configured to rotate about the central axis of the cylindrical basin upon activating the motor.

In some embodiments, the UV-C light tube and the UV-C light strips are configured to emit light having a wavelength between 100 nm and 400 nm. The UV-C light tube and the UV-C light strips can be configured to apply 20-200 mJ/cm³ of UV-C light to the powdered food product to create sterilized powdered food. In some embodiments, the sterilization chamber also includes a push plate having a shape complementary to the cylindrical basin and a push rod configured to move the push plate, where the push rod and push plate are together configured move the powdered food product or sterilized food product out of the cylindrical basin through the door on the first end wall.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a flowchart of a method of the inventive subject matter.

FIG. 2 shows a system of the inventive subject matter incorporating spray drying.

FIG. 3 shows an embodiment of a sterilization chamber configured to fluidize powder.

FIG. 4 shows a sterilization system of the inventive subject matter that incorporates freeze drying.

FIG. 5A shows an embodiment of the inventive subject matter that incorporates freeze drying.

FIG. 5B shows different light or blade shapes.

FIG. 6 shows another sterilization chamber of the inventive subject matter.

FIG. 7 shows another sterilization chamber of the inventive subject matter.

FIG. 8 shows an example screw feature.

FIG. 9 shows another example screw feature.

FIG. 10 shows another sterilization chamber of the inventive subject matter.

FIG. 11 shows an exploded view thereof.

FIG. 12 shows a side, cutaway view thereof.

FIG. 13 shows a front, cutaway view thereof.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used in the description in this application and throughout the claims that follow, the meaning of “a,” “an,” and “the” includes plural reference unless the context clearly dictates otherwise. Also, as used in the description in this application, the meaning of “in” includes “in” and “on” unless the context clearly dictates otherwise.

Also, as used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth in this application should be interpreted as being inclusive of their endpoints and open-ended ranges should be interpreted to include only commercially practical values. Similarly, all lists of values should be considered as inclusive of intermediate values unless the context indicates the contrary.

Although this application is primarily directed to milk and, specifically, human breast milk, its applicable ranges far beyond these uses. Systems and methods of the inventive subject can be used to sterilize any powderized food product without deviating from the inventive subject matter. The detailed description included below focuses on breast milk for illustrative purposes.

In the dairy industry, different techniques have been developed to pasteurize and otherwise sanitize milk. But these relatively commonplace processes are often inadequate for breast milk and other milks that require maximum nutrients preservation. It is often advantageous to powderize milk so that it can be preserved for long periods of time, but ordinary powdered milk is pasteurized before it is powderized, which can diminish the nutritional content of the milk. Embodiments of the inventive subject matter solve this problem by powderizing breast milk first, and then sanitizing the powderized milk using ultra-violet light (e.g., UV-C light), thereby maintaining as much of the milk's nutritional content as possible.

Furthermore, the application utilizes the example of donor breast milk (commonly used in neonatal intensive care units) as a substance with which the invention may be used to elucidate its role in sterilizing a substance without damaging its sensitive nutrients (e.g., sIgA, lactoferrin, and lysozyme among others). This should not be construed as an intention to limit its applicability to other substances in the dairy, food, or biopharmaceutical industries (e.g., human plasma).

Embodiments of the inventive subject matter facilitate meeting safety, nutrition retention, and affordability requirements for donor breast milk to become the best possible option for infants in need. Breast milk subject to treatment by systems of the inventive subject matter is rendered shelf stable via, e.g., powderization (e.g., lyophilization) and UV-C pasteurization.

Systems and methods of the inventive subject matter can result in powdered breast milk being UV-C pasteurized (e.g., under air-fluidized conditions). Research overwhelmingly indicates exposure to UV-C is an effective method for gentle pasteurization of breast milk. This, in part, can depend on the wavelength of UV-C light that is used. Different wavelengths of UV-C light have different effects on biological compounds. Some wavelengths (e.g., 250-298 nm) favor induction of reactive oxygen species (ROS) from aromatic side chains of some amino acids (e.g., tyrosine). This can lead to ROS mediated protein degradation, which is a mechanism of action for eliminating bacteria and inactivating viruses. Other wavelengths can selectively target nucleic acids, causing thymine dimers and other damage to the DNA superstructure to the point it overwhelms the native repair machinery. This selective targeting of nucleic acids is key for UV-C to work as a gentle pasteurization implement: the DNA/RNA material of bacteria and viruses is rendered inert while the immunoglobulins and other immunoprotective proteins are less affected.

Heretofore, UV-C has only been used on breast milk and dairy in liquid form. But when milk is a liquid, it is virtually impossible to expose all the liquid milk in a batch to UV-C light due to the “shadow effect.” The “shadow effect,” as briefly mentioned above, refers to a phenomenon that has largely limited the use of UV light to sterilize surfaces and transparent liquids. Because, e.g., UV-C is a light, any opaque substance it touches casts a shadow on anything beneath it. Therefore, if UV-C is aimed at a heap of powder it would effectively sterilize only a top layer. A similar effect is seen with liquid milk due to its opacity, necessitating UV-C sterilization while vigorously stirring or while spreading milk into extremely thin layers. UV-C is therefore not typically used on powdered breast milk. Embodiments of the inventive subject matter solve this problem by functionally fluidizing powdered milk with sterile air. Air-suspended, constantly circulating particles within a closed, transparent environment can be effectively UV-C pasteurized.

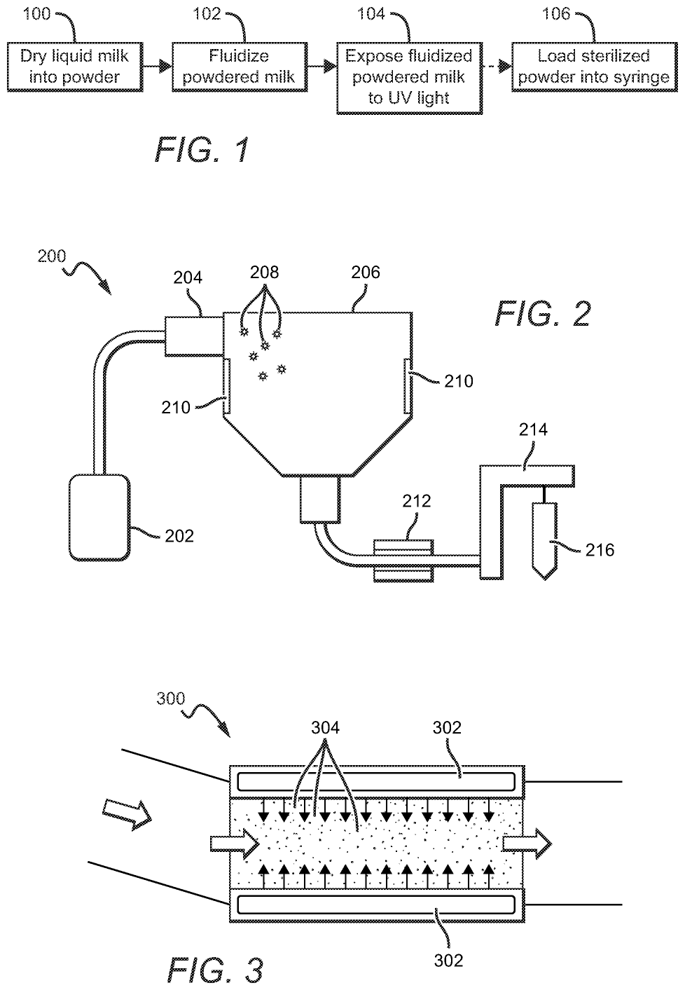

Methods of the inventive subject matter include several steps, which are shown in FIG. 1. In step 100, unpasteurized liquid milk is dried so that it can be powderized. The drying process can be accomplished either by freeze drying or by spray drying the milk. In some embodiments, spray dried milk is introduced into a vat from the drying system, while in other embodiments, a food product (e.g., breast milk) can be dried in one location before it is shipped to a sanitizing facility implementing systems of the inventive subject matter. Thus, although FIGS. 1 and 2 both show an attached drying mechanism (spray dryer 204 and freeze dryer 404), these aspects of the system can be excluded, and powdered food product can be added directly to a system that begins with a vat.

In some embodiments, the vat incorporates UV lights that immediately begin the sanitization process. Next, in step 102, the powdered milk is fluidized so that it can be further sanitized. Fluidization is a process like liquefaction whereby a granular material is converted from a static solid-like state to a dynamic fluid-like state. This process occurs when a fluid (liquid or gas) is passed up through the granular material. Fluidization of the powdered milk causes the powdered milk to mix during step 104, which exposes the fluidized powdered milk to UV light to sanitize the powdered milk (e.g., fluidized milk powder is exposed to UV light to sanitize the powder). In addition, or in the alternative, to exposing powderized milk to UV light during fluidization, powderized milk can also be exposed to UV light by stirring a UV light through the powderized milk, e.g., after the fluidized powder is exposed to UV light. In some embodiments, UV light exposure begins immediately after milk is powderized and before fluidization begins.

During step 104, the fluidized milk is sanitized without significantly diminishing the nutritional content of the milk. Maintaining nutritional value is critical for breast milk to ensure that the babies and children consuming the milk are getting all the nutrition their bodies need to grow and develop. In some embodiments, after the powderized milk is sanitized, it is loaded into syringes according to step 106. Syringes loaded with powdered, sanitized breast milk, can then be distributed. Various systems and devices that can be used to carry out steps of the flowchart shown in FIG. 1 follow.

FIG. 2 shows a system 200 of the inventive subject matter that implements spray drying. Unpasteurized milk is added into the storage area 202 where it can be stored and subsequently pumped to a spray dryer 204. The spray dryer 204 spray dries milk from the storage area 202 into vat 206, such that the spray dried milk has been powderized. Once in vat 206, spray dried milk 208 is exposed to UV light from lights 210. In some embodiments, lights 210 are on when spray dried milk first enters vat 206, and in some embodiments, lights 210 can be turned on once all spray dried milk has entered vat 206. In embodiments where light 210 are turned on and emitting UV light as spray dried milk 208 enters vat 206, the entire batch of spray dried milk 208 is exposed to UV light continuously as it enters vat 206.

Durations of time that powderized milk (e.g., spray dried milk 208) should remain in a vat before moving to sterilization chamber can depend on several factors, including intensity of UV light applied to the powder. For example, exposure of powderized milk to between 20 mJ/cm² to 200 mJ/cm² can result in sufficient sterilization, and recent studies have even shown exposure to between 5 mJ/cm² to 400 mJ/cm² can similarly result in sufficient sterilization. Mixing using compressed air can be implemented to reach these exposure levels. In some embodiments, vat 206 is not the primary vessel for sterilization and no sterilization requirement is imposed before powdered milk can exit.

Lights 210 disposed in vat 206 (or in any device or component of systems of the inventive subject matter featuring lights) can be configured to emit ultraviolet radiation into the vat 206. Different wavelengths of UV radiation are considered in this application, including ultraviolet A (400-315 nm), ultraviolet B (315-280 nm), ultraviolet C (280-100 nm), near ultraviolet (400-300 nm), middle ultraviolet (300-200 nm), far ultraviolet (200-122 nm), hydrogen Lyman-alpha (122-121 nm), vacuum ultraviolet (200-10 nm), and extreme ultraviolet (121-10 nm). Thus, lights 210 can emit radiation at or within any of these ranges, depending on a desired outcome. For example, ultraviolet light having a wavelength between 220 nm and 280 nm (or, more broadly, between 200 and 400 nm) has been found to be effective at breaking down undesirable bacteria and the like while simultaneously maintaining most of the milk's nutritional value. In this way, spray dried milk can be sterilized with minimal impact on the milk's nutritional value. Wavelength ranges described in this paragraph are applicable to all light described in this application, and although the ranges above have different cutoff values, it is contemplated that a range spanning multiple ranges, or portions of ranges, described above can be implemented into embodiments of the inventive subject matter.

To ensure powderized milk is sufficiently exposed to UV light, powderized milk from vat 206 is next passed through a sterilization chamber 212. Sterilization chamber 212 blows air (e.g., compressed air) into its interior while spray dried milk is contained therein. Sterilization chamber 212 can be configured as any one of the sterilization chambers described in this application, and embodiments of the inventive subject matter can include multiple sterilization chambers featuring more than one of the sterilization chambers described in this application, where multiple sterilization chambers are coupled to one another in sequence.

FIG. 3 shows an example sterilization chamber 300 having lights 302 and featuring powder fluidization. Air, shown as arrows pointing inward from the top and bottom of the sterilization chamber 300, is introduced into the sterilization chamber 300 by holes in the walls of the chamber. Introduction of compressed air (e.g., air can be introduced in as a turbulent flow, a laminar flow, or as a flow that begins laminar and transitions to turbulent) fluidizes the powdered milk 304, which is exposed to UV light from lights 302 for as long as needed to ensure that all the powdered milk is sufficiently sanitized. Holes on the bottom of sterilization chamber 300 can be evenly spaced across the interior bottom surface of sterilization chamber 300. Sterilization chamber 300 acts as a passthrough sterilizing device, such that powdered milk is never kept there, instead it passes through and is sterilized as it does so. Thus, sterilization chamber 300 can be made longer or shorter to impact sterilization time as needed.

In some embodiments, lights 302 can be configured to have the same characteristics (e.g., wavelength emissions, etc.) as lights 210, described above. In some embodiments, lights 302 can expose powderized milk 304 to a different wavelength or set of wavelengths of light than lights in an associated vat expose the powderized milk to. In some embodiments, sterilization chamber 300 can include a single light (e.g., just top light 302 as shown in FIG. 3). Whether one or more lights 302 are included, lights 302 can be configured to be stationary relative, or in some embodiments, lights 302 can be configured to move through or around the sterilization chamber 300. Powderized milk 304 can then exit sterilization chamber 300. Thus, once the powderized milk 208 has passed through the sterilization chamber 212, it has been sterilized and is ready to be loaded by a loading mechanism 214 into a syringe 216.

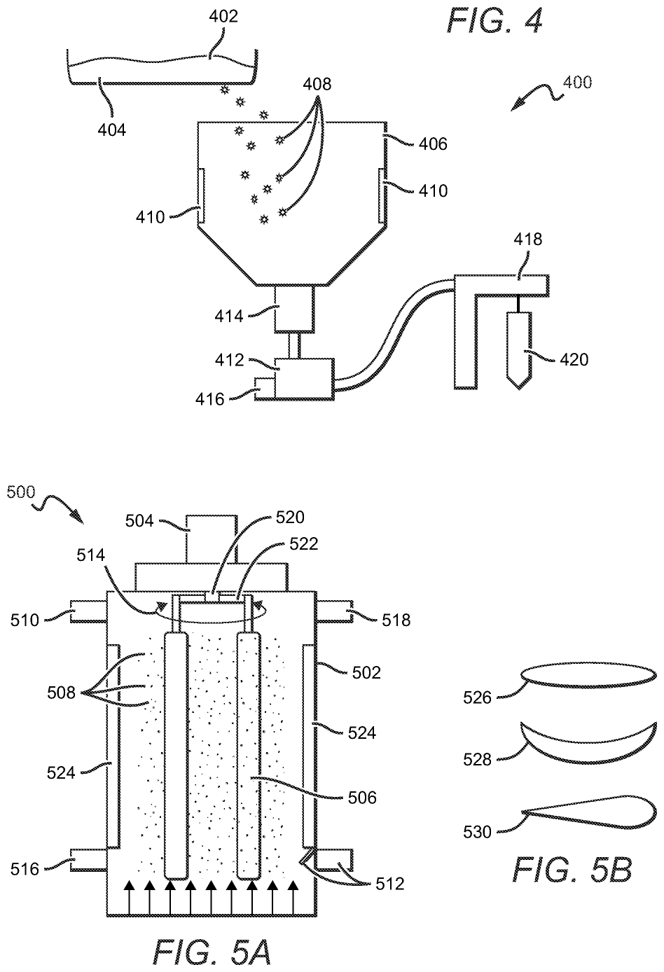

FIG. 4 shows another system 400 of the inventive subject matter that implements freeze drying as a first step as opposed to spray drying as shown above in FIG. 2. Liquid milk 402 is added to a freeze dryer 404, and once it has been freeze dried, it is transferred into vat 406. Vat 406 can be the same vat described above regarding FIG. 2, but, in some embodiments, it can include different features based on its use in association with freeze dried (e.g., powderized) milk 408. Vat 406 thus receivers powderized milk 408 from the freeze drier 404 and begins sterilizing milk using one or more lights 410 disposed therein.

Vat 406, as mentioned above, can feature one or more lights 410 (two lights are depicted in the Figure). These lights can be moving or non-moving, and, as powderized milk 408 is added to vat 406, the first steps of sterilization immediately begin. In some embodiments, vat 406 acts only as a gravity feeding funnel that passes powderized milk into a sterilization chamber 412. Sterilization chamber 412 can be any one of the sterilization chambers described in this application. In some embodiments, sterilization chamber 412 can include more than one of the sterilization chambers described in this application, where multiple sterilization chambers are coupled to one another in sequence. To improve movement of powderized milk 408 into sterilization chamber 412, vat 406 can be agitated (e.g., by component 414, which can be configured to vibrate, introduce pressurized air into vat 406, etc., to motivate movement of powderized milk into sterilization chamber 412).

Sterilization chamber 412 can be configured in a variety of ways. In some embodiments, sterilization chamber 412 can be configured as a sterilization chamber, described above regarding FIG. 2. Compressed air is introduced into sterilization chamber 412 by inlet 416 (e.g., an inlet for compressed air that also includes a compressor, or, in some embodiments, an inlet that couples with a separate compressor). Alternative configurations are shown in FIGS. 5-7. FIG. 5 shows a sterilization chamber 500 having a drum 502, a stirring mechanism 504, and at least one light source 506. Powderized milk 508 enters drum 502 via powder inlet 510 (e.g., by gravity feed or air jet) and exits via powder outlet 512.

Once powdered milk 508 is inside drum 502, it is agitated while exposed to UV light from light source 506. Agitation can be accomplished in a variety of ways, and the embodiment in FIG. 5 relies on at least stirring and fluidization for agitation. As shown by double sided arrow 514, stirring mechanism 504 causes light 506 to move within drum 502, thereby agitating the powderized milk 508 and exposing more of that powderized milk 508 to sterilizing UV light.

In addition to stirring, compressed air is injected into drum 502 according to arrows coming up from the bottom of the drum as shown in FIG. 5A. Compressed air enters drum 502 via compressed air inlet 516 and excess pressure can be released by pressure release valve 518. Compressed air ranging from 10-150 psi can be introduced via inlet 516 and it can be used both to fluidize the powderized milk 508 (e.g., by shooting air upward into drum 502 according to the lines at the bottom of drum 502) and also to move the now-sterilized powderized milk out of drum 502 and into a syringe loading device. To promote mixing, inlet 510 can cause powderized milk 508 to enter drum 502 at an angle to cause it to swirl within drum 502 as it enters where it is further fluidized by compressed air entering the drum from the bottom.

Stirring is caused by stirring mechanism 504, causes light 506 to turn through the powder 508 to bring about more even exposure of UV light to the powder 508. In some embodiments, stirring mechanism includes an electronic motor and can be electronically controlled. Rotation of light 506 can occur between 100 and 3,000 rpm. Stirring speed can be based on, e.g., the amount of powderized milk added to the drum, and in embodiments featuring a controller, programs can be implemented to cause the light 506 to turn at different speeds, even changing speed in the course of mixing a single batch depending on factors such as how much of a batch has already been added to drum 502.

As shown in FIG. 5A, light 506 is coupled with stirring mechanism 504 at the top of drum 502. In some embodiments, a rotor 520 protrudes through the top of drum 502. Rotor 520 can directly couple with a motor in stirring mechanism 504, or, in some embodiments, rotor 520 couples with a gearbox that is in turn coupled with a motor in stirring mechanism 504. It is also contemplated that a motor can be remotely located and stirring mechanism can instead receive mechanical energy by belt, chain, rotating shaft, etc. Light 506 thus couples with rotor 520 by arm 522. In some embodiments, arm 522 extends the length of light 506, creating a fin that encourages mixing when light 506 turns through drum 502. In some embodiments, a second light can be included opposite light 506 (e.g., as shown in FIG. 5A). Additional lights can also be included beyond just the two shown without departing from the inventive subject matter. More lights result in greater exposure of powderized milk to UV light, which can decrease sterilization times. In some embodiments, instead of a second light, a second stirring component opposite light 506 can be included instead of a second light, where the second stirring component facilitates stirring.

In some embodiments, light 506 (and any other light like it) extends from arm 522 down to near the bottom of drum 502. In some embodiments, the bottom portion of light 506 extends down to within a half inch of the bottom of drum 502, leaving enough room for air to come up from the bottom of drum 502 to fluidize the powderized milk therein. The bottom surface of light 506 (and any other matching light, as shown in FIG. 5A) can come within 0.25-0.5″, 0.5-1″, 1″-2″, or 3-5″ of the bottom, interior surface of drum 502. Other distances are contemplated depending on the internal volume of drum 502.

In some embodiments, light 506 (and any other light or stirring component disposed within drum 502) can be configured as a blade (e.g., as a blade without lights incorporated therein, or a blade with lights incorporated therein). FIG. 5B shows a variety of different blade cross sections including an oval 526, a crescent 528, and an air foil 530. Additionally or alternatively to lights incorporated into stirring mechanisms (e.g., light 506), lights can be included within the drum, shown as lights 524 in FIG. 5A. In embodiments where lights 524 are included in the drum and lights are not included in either stirring component (e.g., light 506 is no longer a light, nor is its complementary component shown in FIG. 5B), stirring components can be made from UV resistant material to improve their useful lifetimes and minimize wear and tear. In similar embodiments, stirring components can be made from a reflective material, a transparent material (e.g., transparent to all or some UV radiation wavelengths discussed in this application, preferably at least transparent to wavelengths of UV light emitted from lights incorporated into a sterilization chamber), or translucent material (or any combination thereof) to improve exposure of powderized milk to UV light from lights 524. Although two lights 524 are shown, it is contemplated that any number of lights can be included without departing from the inventive subject matter.

Once powderized milk 508 is sufficiently sterilized it can be pushed out powder outlet 512. Powder outlet 512 can include a door 532 that opens to allow sterilized powderized milk 508 to be pushed out of drum 502. Pressure from incoming air pushes the sterilized powderized milk out through powder outlet 512. To avoid over pressurization, which could lead to explosion, pressure relief valve 518, which can feature an air filter, is located at the top of drum 502. In embodiments with an air filter, the air filter allows air to escape without allowing powder to escape.

Thus, returning to FIG. 4, compressed air enters the sterilization chamber 412 via inlet 416 and, after the powderized milk is sterilized, compressed air from inlet 416 pushes the sterilized and powderized milk to syringe loading mechanism 418 where it can be packaged into a syringe 420. In some embodiments, pressure from air entering and subsequently existing the sterilization chamber 412 pushes powderized milk through syringe loading mechanism 418 and into a syringe 420.

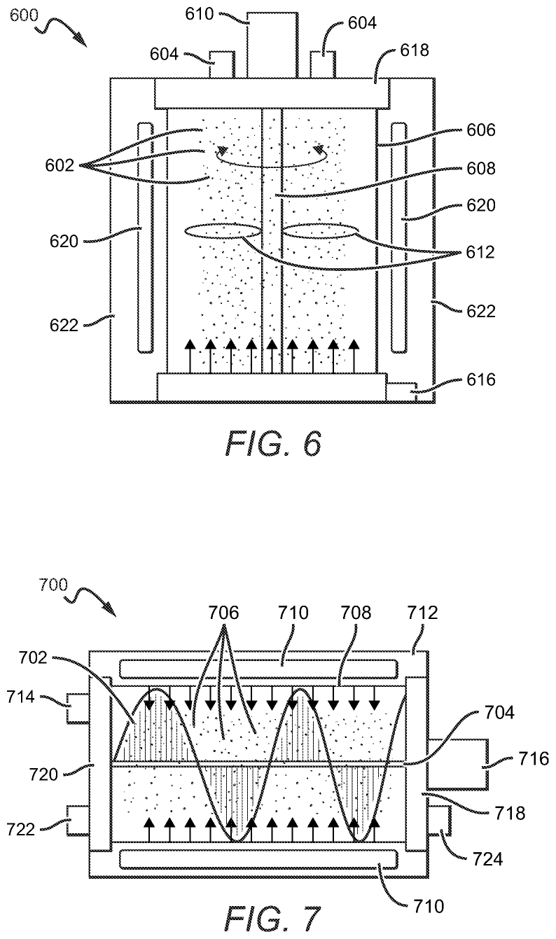

FIG. 6 shows another sterilization chamber 600 of the inventive subject matter. Sterilization chamber 600 receives powderized milk 602 via inlet 604 into drum 606. In some embodiments, drum 606 is configured to have a cylindrical interior. Powderized milk can be gravity fed into drum 606 or, in some embodiments, it can be pushed into drum 606 by pressurized air. Once powderized milk 602 is contained within drum 606, shaft 608 is rotated by motor 610. In some embodiments, motor 610 can instead be a rotational drive mechanism that receives remotely generated mechanical energy, such as a gearbox, a pulley or pulley system, etc. Motor 610 causes shaft 608 to rotate, and fan blades 612 that are coupled with shaft 608 thereby rotate, causing the powderized milk 602 to circulate within drum 606. Shaft 608 is attached at the top to motor 610 and it can be coupled with the bottom of drum 606 by a bearing or other low-friction coupling.

Motor 610 can be a high torque, high RPM (e.g., 5,000-20,000) electric motor such as a DC motor or a universal motor (e.g., one that can operate on AC or DC voltage). Other electric motors can also be implemented without deviating from the inventive subject matter. In some embodiments, motor 610 includes a gearbox that is coupled between motor 610 and shaft 608. Causing powderized milk 602 to circulate within drum 606 improves its exposure to sterilizing UV light.

Inlet 614 can be used to introduce pressurized air. Pressurized air from inlet 614 can be introduced for several functions. In some embodiments, it is used to introduce air via holes—shown as upward pointing arrows—on the bottom of the interior of drum 606. In some embodiments, pressurized air entering via inlet 614 is used to push powderized milk 602 out through outlet 616, which couples with a syringe loader as shown in FIG. 4 (e.g., where sterilization chamber 600 takes the place of sterilization chamber 412). When integrated into a system such as the one shown in FIG. 4, inlet 416 couples with, or is the same component as, inlet 614. In some embodiments, air holes do not exist on the bottom of drum 606, and sterilization chamber 600 uses only fan blades 612 to circulate the powderized milk 602 within drum 606. Although two fan blades are shown, additional fan blades can be added without deviating from the inventive subject matter.

Sterilization chamber 600 can feature a removable top 618 (e.g., a top that is configured to screw on, to clip on, or to otherwise be coupled with a top portion of drum 606 without the use of an adhesive or other fastening mechanism that would result in damage upon removal). Top 618 includes holes at least for inlet 604, motor 610, and inlet 614.

In some embodiments, drum 606 features transparent (e.g., transparent to all or some UV radiation wavelengths discussed in this application, preferable at least transparent to UV wavelengths emitted by lights a sterilization chamber) walls where lights 620 are disposed outside drum 606. As powderized milk 602 is mixed by rotating blades 612 attached to shaft 608, it is sterilized by UV radiation emitted from lights 620. Although lights 620 are drawn to show two lights, any number of lights can be used to improve exposure of powderized milk 602 to UV light. In some embodiments, lights 620 are disposed outside of drum 606, and lights 620 can be configured in elongated packages (e.g., like fluorescent lights or LED light strips) with multiple lights surrounding the entirety of drum 606. Before powderized milk 602 is introduced into drum 606, and thus before blades 612 rotate, top 618 is coupled with drum 606 to create an air-tight seal. Drum 606 is also sealed around all other coupling points to prevent unwanted air from exiting or entering.

Components disposed within drum 606 can be made from reflective materials such as aluminum (e.g., polished aluminum) to reflect UV light to improve exposure of powderized milk 602 to sterilizing UV light. Moreover, drum 606 can be surrounded by a reflective shield 622. Reflective shield 622 can be configured as a cylinder to surround drum 606, thus reflecting UV light that would otherwise project away from drum 606 back into drum 606. In some embodiments, lights 620 are integrated into shield 622. In some embodiments, lights 620 are integrated into the interior of drum 606 like what is shown in sterilizing chamber 500. In any embodiment discussed in this application, UV lights can be integrated into the walls of a sterilization chamber's drum to improve efficiency using, e.g., UV LEDs. In such embodiments, the interior of drum 606 can include a reflective material. Lights 620 can be configured to project light at any wavelength discussed in this application, preferably between 248 and 310 nm.

Sterilization chamber 600 additionally includes a bottom cap 624. Bottom cap 624 is configured to hold a bottom portion of shaft 608 in place (e.g., so that it is vertically oriented as shown in FIG. 6). In some embodiments, powder outlet 616 couples with bottom cap 624. It is also contemplated that powder outlet 616 can alternatively couple with a wall of drum 606. Once powderized milk 602 has been exposed to UV light long enough to become sufficiently sterilized, it can exit drum 606 via outlet 616, e.g., by introducing compressed air to the drum to push the powderized milk out. In some embodiments, compressed air can push sterilized milk powder from the sterilization chamber into a syringe loading device, as shown in, e.g., FIG. 4.

Another sterilization chamber of the inventive subject matter is shown in FIG. 7. Sterilization chamber 700 relies primarily on a screw-type agitation system, featuring screw feature 702 that is fixed to shaft 704. As shaft 704 rotates, screw 702 turns causing powderized milk 706 to be stirred within drum 708 (e.g., powderized milk can be caused to move to the walls of drum 708, thereby exposing the powderized milk to UV light shining into drum 708). Lights 710 are disposed outside of drum 708. Although two lights are shown in FIG. 7, external light configurations shown in FIG. 7 can be the same as those described regarding FIG. 6. For example, sterilization chamber 700 can include a reflective shield 712 surrounding lights 710, and lights 710 can be numerous and configured to surround drum 708, and, additionally or alternatively, lights can be integrated into reflective shield 712. Drum 708 thus includes walls that comprise materials that are at least partially (in some embodiments, fully) transparent to UV radiation corresponding to the wavelengths of UV light emitted from lights 710 (e.g., any of the wavelengths discussed in this application, and preferably 248 to 310 nm).

In some embodiments, motor 716 includes with a gearbox that is in turn coupled with shaft 704. It is also contemplated that a motor can be remotely located and instead, motor 716 can be a device or system that is configured to receive mechanical energy by belt, chain, rotating shaft, etc. Regardless of configuration, shaft 704 can be configured to rotate between 100 and 1000 RPM. Rotating screw feature 702 can create air currents and other interactions with powderized milk 706 that can be used to motivate powderized milk 706 out of drum 708 via outlet 724 and toward a syringe loading device as shown in FIG. 4. Compressed air introduced via inlet 722 can additionally or alternatively push powderized milk toward such a syringe loading device.

In some embodiments, compressed air can be introduced into drum 708 as demonstrated by arrows pointing inward from the interior walls of drum 708. This can facilitate additional mixing of powderized milk 706 to improve its exposure to sterilizing UV light from lights 710. Powderized milk 706 can enter drum 708 via inlet 714, which can be closed during the mixing and sterilization process. End cap 720 features through holes for both powder inlet 714 as well as inlet 722 for compressed air to enter drum 708. Shaft 704 is coupled with motor 716 such that motor 716 can cause shaft 704 to turn, thereby turning screw feature 702. Mechanical energy passes through end cap 718, which couples with drum 708 to create a seal that powderized milk 706 cannot escape through. End cap 720 can include a bearing (e.g., a ball bearing, a friction bearing, etc.) for shaft 704 to couple with to keep it from moving off axis and to facilitate rotation.

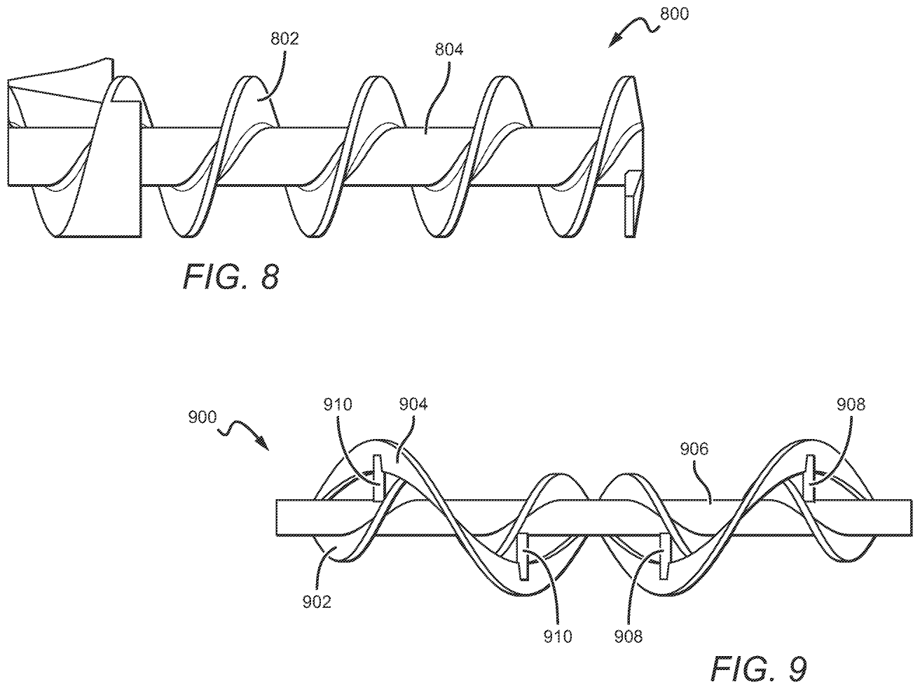

FIGS. 8 and 9 show example screw features that can be used in the sterilization chamber of FIG. 7. Screw feature 800 shows a simple screw having a single fin 802 that spirals around a shaft 804 from one end to another. Screw feature 900 features two fins 902 and 904 that spiral around a shaft 906. Fin 902 beings in contact with shaft 906 and around the middle of shaft 906 an air gap separates fin 902 from shaft 906 with arms 908 to give fin 902 stability and such that fin 904 can spiral between fin 902 and shaft 906. Fin 904 spirals in an opposite direction from fin 902, beginning with separation from shaft 906. Fin 904 is held separate from shaft 906 by arms 910 such that fin 902 can spiral between fin 904 and shaft 906. Around the middle of shaft 906, fin 904 transitions such that it is in contact with shaft 906.

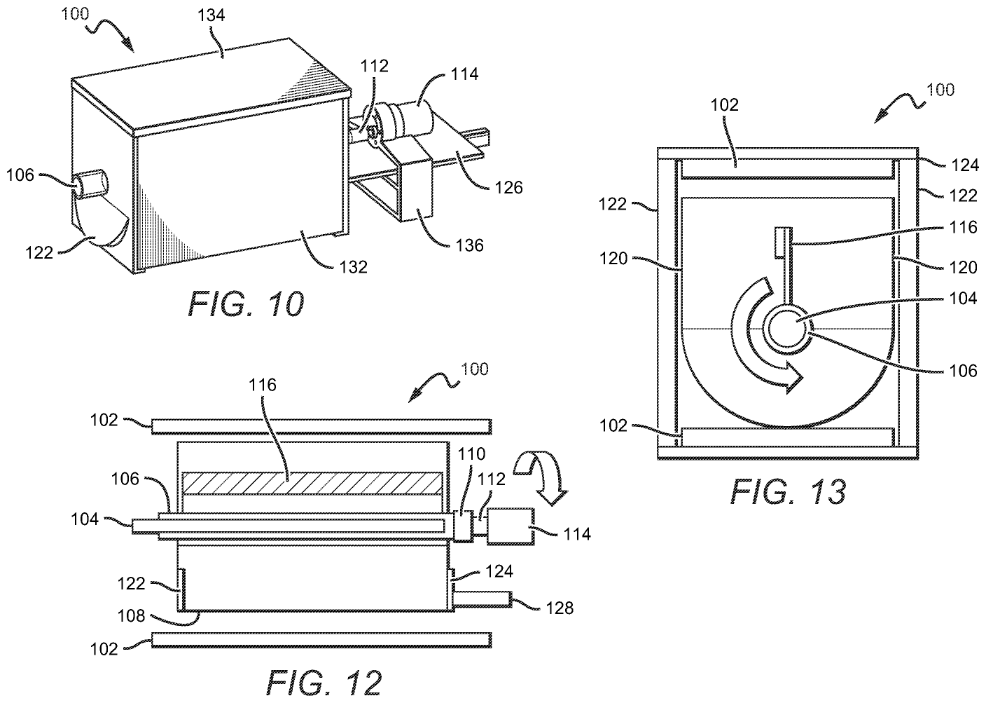

FIG. 10 shows another embodiment of a sterilization chamber 1000 of the inventive subject matter. Chamber 1000 features a variety of different features, aspects, and components that facilitate UV sterilization of, e.g., powderized milk. Sterilization chamber 1000 can be implemented in any portion of a system of the inventive subject matter described in this application described above as accommodating such a sterilization chamber (see, e.g., FIGS. 3, 5A, and 6-7).

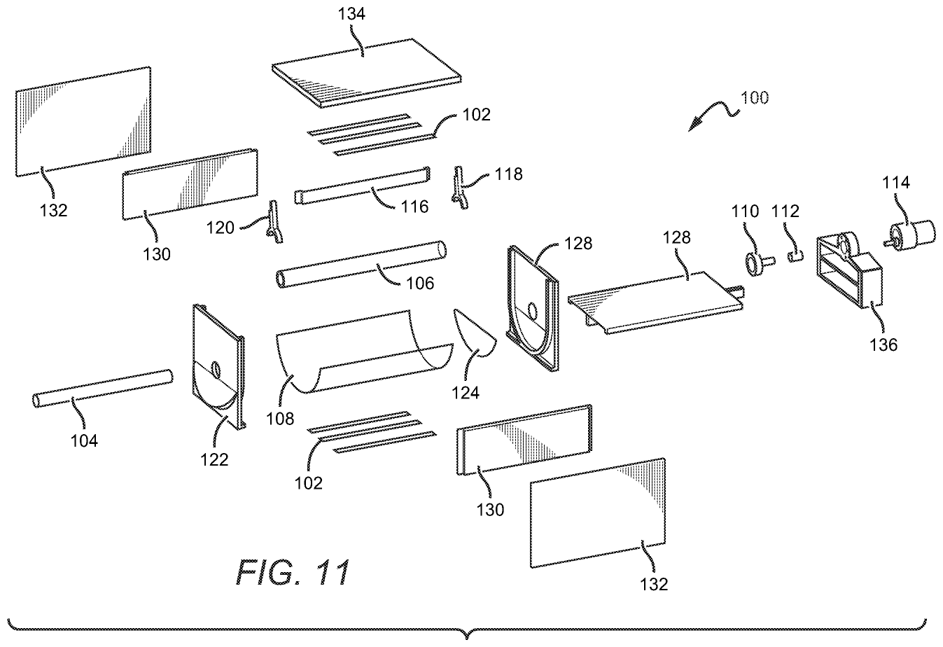

Sterilization chamber 1000 comprises many different components that make its functionality possible. FIG. 11, which shows an exploded view sterilization chamber 1000, shows each of these components, including: a UV-C light strips 1002, a UV-C light tube 1004, a transparent tube 1006, a cylindrical basin 1008, a tube connector 1010, a shaft coupling 1012, a motor 1014, a paddle ribbon 1016, a paddle right end 1018, a paddle left end 1020, an emptying end-wall 1022, a push plate 1024, a push plate end wall 1026, a push rod 1028, inner side-walls 1030, outer side-walls 1032, a lid 1034, and a motor stand 1036. Transparent tube 1006 and cylindrical basin 1008 can be made from any suitable, transparent material that allows UV light to pass through it, such as glass, plastic, or a combination thereof. Materials for these components that block UV light could render the device inoperable, so minimal UV blocking is desirable. Quartz glass is contemplated as an appropriate material for transparent tube 1006 and cylindrical basin 1008. UV-C light emitters can be configured in a variety of different ways. For example, UV-C light strips can be configured as tubular light emitters, etc.

In some embodiments, powdered milk (or other powdered food product) enters sterilization chamber 1000 by lifting lid 1034 so that an operator can pour the powdered product inside. In some embodiments, this process can be automated using actuators to lift lid 1034 to allow for the powderizing components of a system to automatically introduce powdered milk to sterilization chamber 1000. Once lid 1034 is closed, paddle ribbon 1016 rotates to toss and mix powder contained within sterilization chamber 1000. During mixing, UV-C light strips 1002 shine from beneath and above a mixing cavity (formed by, e.g., cylindrical basin 1008, inner side-walls 1030 as well as lid 1034), along with light from UV-C light tube 1004, which together expose powder within the sterilization chamber 1000 to UV-C light that sterilizes the powder. Mixing and UV-C exposure can go for as long as necessary to reduce bacteria levels. All discussion included above regarding UV-C lights, exposure, mixing, etc. can also be applied to sterilization chamber 1000. It is expressly contemplated that any aspect of the systems described above in this application can be adapted to operate with sterilization chamber 1000.

Paddle ribbon 1016 is rotated by motor 1014. Motor 1014 has an output shaft that couples with shaft coupling 1012 that in turn couples with tube connector 1010. Tube connector 1010 couples with paddle right end 1018. Paddle right end 1018 couples with a right end of paddle ribbon 1016 and paddle left end 1020 couples with a left end of paddle ribbon 1016. Thus, causing motor 1014 to turn causes paddle ribbon 1016 to rotate within the sterilization chamber.

Inner side-walls 1030 are configured to extend upward from top edges of cylindrical basin 1008 to help to create a continuous inner surface of the mixing cavity. It is contemplated that these inner walls can be made from a UV-transparent material or a UV-opaque material. In some embodiments, the inner side-walls 1030 can feature a reflective inward-facing surface or have a reflective coating applied to an inward-facing surface. Outer side-walls 1032 can be made from a material having a reflective inner surface or a reflective coating on its inner surface, as well. Reflective inner surfaces of either the inner side-walls 1030 or the outer side-walls 1032 can improve UV-C sterilization that occurs within sterilization chamber 1000 by reflecting UV-C light within the chamber, causing it to reach powdered food product from angles that it otherwise might not. This can reduce an amount of time that powdered food product needs to remain in sterilization chamber 1000 before it reaches a desired degree of sanitization.

Once the mixing is complete (e.g., the powder within the mixing cavity is sterilized to a desired degree), powder can be pushed from sterilization chamber 1000. First, paddle ribbon 1016 is rotated to a top portion of the mixing cavity and then emptying assembly (which includes at least push plate 1024 and push rod 1028) slides into the mixing cavity. Push plate 1024 is moved by push rod 1028 to push sterilized powder through a door disposed on emptying end-wall 1022. Not all powder may be evacuated in a single push, so emptying assembly can be configured to slide back and forth multiple times. Emptying door and emptying assembly can be operated by, e.g., a motor-controlled rack-and-pinion or an operator moving them manually.

Because UV-C sterilization is a “line-of-sight” technology, sterilization chamber 1000 is configured to agitate, e.g., freeze-dried, human breast milk powder within the main cavity of the device such that all the powder is exposed evenly to UV-C light. UV-C light shines from three points into the main cavity: UV-C light strips shine up from the bottom through cylindrical basin 1008, UV-C light strips shine down from the top, and UV-C light tube 1004 radiates light outward from, e.g., an axis of paddle ribbon 1016 rotation and out of transparent tube 1008. Paddle ribbon 1016 can be configured according to cross-sectional shapes shown in FIG. 5B. It is also contemplated that one or any combination of cross-sectional shapes shown in FIG. 5B can be implemented into paddle ribbon 1016.

FIGS. 12 and 13 shows a side view and a rear view, respectively, of aspects of sterilization chamber 1000, which showcase how various components within sterilization chamber 1000 are arranged and positioned. These views also demonstrate how rotational output from motor 1014 can cause paddle ribbon 1016 to turn within the mixing cavity. These figures also show positioning of UV-C light strips 1002 and UV-C light tube 1004 within the sterilization chamber 1000. These figures also show that UV-C light tube 1004 is positioned along a central axis of the cylindrical basin 1008.

Thus, specific systems and methods of sanitizing powdered milk (e.g., human breast milk) have been disclosed. It should be apparent, however, to those skilled in the art that many more modifications besides those already described are possible without departing from the inventive concepts in this application. The inventive subject matter, therefore, is not to be restricted except in the spirit of the disclosure. Moreover, in interpreting the disclosure all terms should be interpreted in the broadest possible manner consistent with the context. In particular, the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A sterilization chamber for powdered food product comprising:
a cylindrical basin configured to receive the powdered food product;
a first end wall and a second end wall disposed on each end of the cylindrical basin, wherein the first end wall comprises a door;
a transparent tube having a UV-C light tube disposed therein, wherein the transparent tube is positioned along a central axis of the cylindrical basin;
wherein the transparent tube couples with the first end wall and with the second end wall;
a paddle ribbon configured to rotate about the central axis of the cylindrical basin; and
a set of UV-C light strips disposed above the cylindrical basin.

2. The sterilization chamber of claim 1, wherein the UV-C light tube and the UV-C light strips are configured to emit light having a wavelength between 100 nm and 400 nm.

3. The sterilization chamber of claim 1, wherein the UV-C light tube and the UV-C light strips are configured to apply 20-200 mJ/cm³ of UV-C light to the powdered food product to create sterilized powdered food.

4. The sterilization chamber of claim 1, further comprising a push plate having a shape complementary to the cylindrical basin and a push rod configured to move the push plate, wherein the push rod and push plate are together configured move the powdered food product or sterilized food product out of the cylindrical basin through the door on the first end wall.

5. A sterilization chamber for powdered food product comprising:
a cylindrical basin configured to receive the powdered food product;
a first end wall and a second end wall disposed on each end of the cylindrical basin, wherein the first end wall comprises a door;
a lid coupled with the first end wall and the second end wall;
a transparent tube having a UV-C light tube disposed therein, wherein the transparent tube is positioned along a central axis of the cylindrical basin;
wherein the transparent tube couples with the first end wall and with the second end wall;
a set of UV-C light strips disposed above the cylindrical basin; and
a motor coupled with a paddle ribbon, wherein the paddle ribbon is configured to rotate about the central axis of the cylindrical basin upon activating the motor.

6. The sterilization chamber of claim 5, wherein the UV-C light tube and the UV-C light strips are configured to emit light having a wavelength between 100 nm and 400 nm.

7. The sterilization chamber of claim 5, wherein the UV-C light tube and the UV-C light strips are configured to apply 20-200 mJ/cm³ of UV-C light to the powdered food product to create sterilized powdered food.

8. The sterilization chamber of claim 5, further comprising a push plate having a shape complementary to the cylindrical basin and a push rod configured to move the push plate, wherein the push rod and push plate are together configured move the powdered food product or sterilized food product out of the cylindrical basin through the door on the first end wall.
